(19) United States
(12) Patent Application Publication
(10) Pub. No.: US 2023/0134020 A1
(43) Pub. Date: May 4, 2023

(54) OPTICAL DEVICE AND OPERATING METHOD THEREOF

(71) Applicants: Yun-Hsuan LIN, Taipei City (TW); Crystalvue Medical Corporation, Taoyuan City 330 (TW)

(72) Inventors: Yun-Hsuan LIN, Taipei City (TW); William WANG, Taoyuan City (TW); Chung-Cheng CHOU, Luzhu Township (TW)

(21) Appl. No.: 17/967,680

(22) Filed: Oct. 17, 2022

(30) Foreign Application Priority Data
Oct. 29, 2021 (TW) 110140325

(51) Int. Cl. G06F 3/01

(52) U.S. Cl. CPC G06F 3/013; A61B 3/113

(57) ABSTRACT

An optical device including a positioning module, a multi-band light-source module, a tracking and locking module, a monitoring module and a control module is disclosed. The positioning module positions eyes according to their characteristics. The multi-band light source module is coupled to the positioning module. After the positioning module positions eyes, the multi-band light-source module emits multi-band light to eyes. The tracking and locking module tracks and locks eyes and provides first information including whether eyes are locked. The monitoring module monitors eyes and provides second information including whether eyes are emitted by the multi-band light for a default time. The control module is coupled to the tracking and locking module, the monitoring module and the multi-band light-source module to generate a control signal according to the first information and the second information to control the multi-band light-source module to continuously or stop emitting the multi-band light to eyes.

### RELATED APPLICATION

This application claims the benefit of TW Patent Application No. 110140325, filed Oct. 29, 2021, the contents of which are incorporated herein by reference in its entirety.

### BACKGROUND OF THE INVENTION

### 1. Field of the Invention

The invention relates to an optical device; in particular, to an optical device applied to eyes and an operating method thereof.

### 2. Description of the Prior Art

In general, the conventional optical device still has the following shortcomings when actually performing optical treatment on the eye of a patient, which needs to be improved:

(1) it can only scan at a single point;

(2) the light is emitted through the sclera or with the closed eyes, resulting in an incalculable amount of light energy reaching the retina; and

(3) the retina cannot be irradiated with local precise positioning.

### SUMMARY OF THE INVENTION

Therefore, the invention provides an optical device and an operating method thereof to solve the above-mentioned problems of the prior arts.

An embodiment of the invention is an optical device. In this embodiment, the optical device includes a positioning module, a multi-band light-source module, a tracking and locking module, a monitoring module and a control module. The positioning module is configured to position an eye according to characteristics of the eye. The multi-band light-source module is coupled to the positioning module. After the eye is positioned by the positioning module, the multi-band light-source module is configured to emit a multi-band light to the eye. The tracking and locking module is configured to track and lock the eye and provide a first information including whether the eye is locked. The monitoring module is configured to monitor the eye and provide a second information including whether the eye is irradiated by the multi-band light for a default time. The control module is coupled to the tracking and locking module, the monitoring module and the multi-band light-source module and configured to generate a control signal according to the first information and the second information to control the multi-band light-source module to continuously or stop emitting the multi-band light to the eye.

In an embodiment, the optical device is designed as a machine-type optical device.

In an embodiment, the optical device is designed as a portable/wearable optical device. The positioning module, the tracking and locking module and the monitoring module are disposed independently, or integrated/combined with the control module and other mobile platforms.

In an embodiment, when an eyeball of the eye moves slightly and is still in a locked state and the eye is irradiated by the multi-band light for the default time, the control module controls the multi-band light-source module to continuously emit the multi-band light to the eye.

In an embodiment, when an eyeball of the eye moves substantially and is released from a locked state or the eye is irradiated by the multi-band light for the default time, the control module controls the multi-band light-source module to stop emitting the multi-band light to the eye.

In an embodiment, the multi-band light-source module includes a regulating unit and a projection light-source. The regulating unit is configured to control the multi-band light emitted by the projection light-source according to an open state or a closed state of the eye.

In an embodiment, when the eye is in the open state and the projection light-source is watched by the eye, the regulating unit controls the projection light-source to emit the multi-band light on a part or all of the retina of the eye; when the eye is in the open state but the projection light-source is not watched by the eye, the regulating unit controls the projection light-source to project the multi-band light through the sclera of the eye.

In an embodiment, when the eye is in the closed state, the regulating unit controls the projection light-source, close to or contacting the eyelid of the eye, to project the multi-band light.

In an embodiment, an electrochromic layer is attached to the multi-band light source module and the wavelength is changed by changing the driving voltage, or a filter is attached and the wavelength is changed by moving the filter, or the light source is switched by moving an aperture layer.

In an embodiment, the multi-band light source module includes a plurality of light source units distributed on the edge and a polarized light refraction unit to provide light projection treatment areas at different positions.

In an embodiment, when the plurality of light source units is distributed in different height layers, the polarized light refraction unit provides light projection of different wavelengths by height switching.

In an embodiment, when the plurality of light source units is distributed on the same height layer, the polarized light refraction unit provides light projection of different wavelengths by translation or rotation.

In an embodiment, the polarized light refraction unit and the plurality of light source units can be independently driven to provide different scanning modes with different shaped paths.

In an embodiment, the characteristics of the eye are retinal blood vessels.

Another preferred embodiment of the invention is an optical device operating method. In this embodiment, the optical device operating method includes following steps of: (a) positioning an eye according to characteristics of the eye; (b) after the eye is positioned, emitting a multi-band light to the eye; (c) tracking and locking the eye and providing a first information including whether the eye is locked; (d) monitoring the eye and providing a second information including whether the eye is irradiated by the multi-band light for a default time; and (e) generating a control signal according to the first information and the second information to continuously emit the multi-band light to the eye or stop emitting the multi-band light to the eye.

In an embodiment, when an eyeball of the eye moves slightly and is still in a locked state and the eye is irradiated by the multi-band light for the default time, the step (e) continuously emits the multi-band light to the eye.

In an embodiment, when an eyeball of the eye moves substantially and is released from a locked state or the eye is irradiated by the multi-band light for the default time, the step (e) stops emitting the multi-band light to the eye.

In an embodiment, the optical device operating method further includes a step of: regulating the multi-band light emitted by the step (b) according to an open state or a closed state of the eye.

In an embodiment, when the eye is in the open state and the projection light-source is watched by the eye, regulating the step (b) to emit the multi-band light on a part or all of the retina of the eye; when the eye is in the open state but the projection light-source is not watched by the eye, regulating the step (b) to project the multi-band light through the sclera of the eye.

In an embodiment, when the eye is in the closed state, regulating the step (b), close to or contacting the eyelid of the eye, to project the multi-band light.

Compared to the prior art, the optical device and an operating method thereof in the invention can achieve ideal curative effect by projecting lights with different wavelength bands to the fundus of the patient, and can use the retinal blood vessels to locate and track the position change of the patient's eyeball. And, the position of the retina of the irradiation target can still be accurately positioned when moving, and the irradiation can be stopped immediately when the patient's eyeball moves greatly to ensure safety.

The advantage and spirit of the invention may be understood by the following detailed descriptions together with the appended drawings.

### BRIEF DESCRIPTION OF THE APPENDED DRAWINGS

FIG. 1 illustrates a schematic diagram of the optical device applied to the eye in a preferred embodiment of the invention.

FIG. 2 illustrates a flowchart of the optical device operating method applied to the eye in another preferred embodiment of the invention.

FIG. 3 illustrates a schematic diagram that the different light source units distributed on the edge cooperate with the polarized light refraction unit to provide light projection treatment areas at different positions.

FIG. 4 illustrates a schematic diagram that when different light source units are distributed in different height layers, the polarized light refraction unit can provide light projection of different wavelengths by height switching.

FIG. 5 and FIG. 6 illustrate schematic diagrams that when different light source units are distributed on the same height layer, the polarized light refraction unit can provide light projection of different wavelengths by translation or rotation.

FIG. 7 illustrates a schematic diagram of the multi-band light source module with additional electrochromic layer and changing driving voltage, moving additional filter.

FIG. 8 illustrates a schematic diagram of the multi-band light source module using the displacement of the aperture layer to switch the light source.

FIG. 9 illustrates a schematic diagram that both the polarizing refraction unit and the light source unit can be independently driven to provide different scanning modes with different shaped paths.

### DETAILED DESCRIPTION OF THE INVENTION

Exemplary embodiments of the invention are referenced in detail now, and examples of the exemplary embodiments are illustrated in the drawings. Further, the same or similar reference numerals of the components/components in the drawings and the detailed description of the invention are used on behalf of the same or similar parts.

An embodiment of the invention is an optical device. In this embodiment, the optical device can be an optical device used for optical treatment of the eye, which can be designed as a machine-type optical device or a portable/wearable optical device according to the requirements of practical applications, but not limited to this.

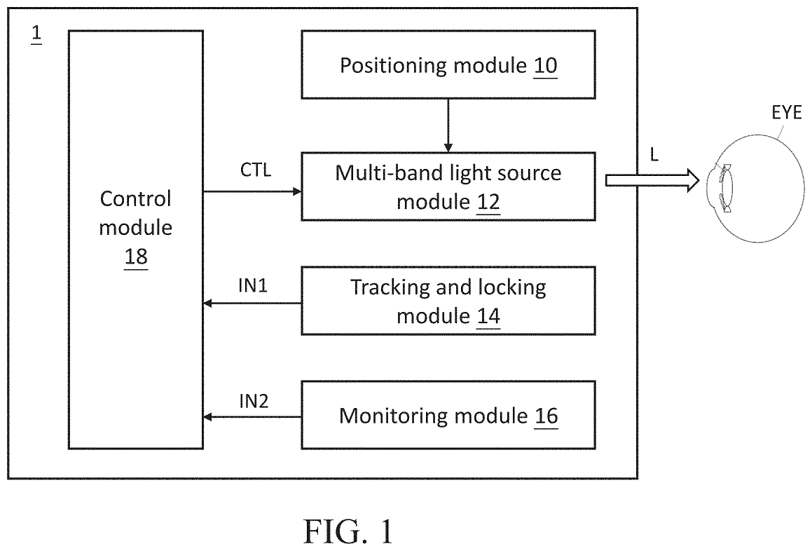

Please refer to FIG. 1. FIG. 1 illustrates a schematic diagram of the optical device applied to the eye in this embodiment. As shown in FIG. 1, the optical device 1 includes a positioning module 10, a multi-band light source module 12, a tracking and locking module 14, a monitoring module 16 and a control module 18. The positioning module 10 is coupled to the multi-band light source module 12. The multi-band light source module 12 is coupled to the control module 18. The tracking and locking module 14 is coupled to the control module 18. The monitoring module 16 is coupled to the control module 18. The control module 18 is coupled to the multi-band light source module 12, the tracking and locking module 14 and the monitoring module 16 respectively.

The positioning module 10 is used for positioning the patient's eye EYE according to eye features (e.g., retinal blood vessels, but not limited to this). After the positioning module 10 completes the positioning of the patient's eye EYE, the multi-band light source module 12 will start to emit a multi-band light L to the patient's eye EYE.

When the multi-band light source module 12 emits the multi-band light L to the patient's eye EYE, the tracking and locking module 14 will track and lock the patient's eye EYE to confirm whether the patient's eye EYE is in the locked state. The tracking locking module 14 will provide the control module 18 with the first information IN1 including whether the patient's eye EYE is in the locked state.

When the multi-band light source module 12 emits the multi-band light L to the patient's eye EYE, the monitoring module 16 will monitor the length of time that the patient's eye EYE is irradiated by the multi-band light L, so as to provide the second information IN2 including whether the patient's eye EYE has been irradiated by the multi-band light L for the default time to the control module 18. In fact, the default time is related to the default optical therapeutic effect to be achieved in this treatment, and the default time can be preset by the system or set by the operator, but not limited to this.

When the control module 18 receives the first information IN1 from the tracking and locking module 14 and receives the second information IN2 from the monitoring module 16, the control module 18 will generate the control signal CTL to the multi-band light source module 12 according to the first information IN1 and the second information IN2, so as to control the multi-band light source module 12 to continuously emit the multi-band light L to the patient's eye EYE, or stop emitting the multi-band light L to the patient's eye EYE.

In an embodiment, if the patient's eye EYE moves only a small amount and the multi-band light L has not reached the default time, the first information IN1 sent by the tracking and locking module 14 will include that the patient's eye EYE is still in the locked state and the second information IN2 sent by the monitoring module 16 will include that the patient's eye EYE has not been irradiated by the multi-band light L for the default time. At this time, the control module 18 will confirm the safety of the patient's eye EYE receiving optical irradiation according to the first information IN1 and the second information IN2, and determine that the default optical therapeutic effect has not yet reached; therefore, the control module 18 will emit the control signal CTL to control the multi-band light source module 12 to continuously emit the multi-band light L to the patient's eye EYE.

In another embodiment, if the patient's eye EYE has largely moved and released from the locked state, the first information IN1 sent by the tracking and locking module 14 will include that the patient's eye EYE has been released from the locked state. At this time, in order to prevent the patient's eye EYE from being hurt, the control module 18 will immediately send a control signal CTL to control the multi-band light source module 12 to stop emitting the multi-band light L to the patient's eye EYE, so as to ensure the security of patient's eye EYE. In fact, the small movement and the large movement are determined according to the threshold movement distance, and the threshold movement distance can be preset by the system or set by the operator, but not limited to this.

In another embodiment, if the patient's eye EYE has been irradiated by the multi-band light L for a default time, then the second information IN2 sent by the monitoring module 16 will include that the patient's eye EYE has been irradiated with multi-band light L for a default time. At this time, the control module 18 will immediately send the control signal CTL to control the multi-band light source module 12 to stop emitting the multi-band light L to the patient's eye EYE, so as to end the treatment.

In practical applications, the multi-band light source module 12 can include a regulating unit and a projection light source. The regulating unit can regulate the projection light source to emit the multi-band light L according to the open state or the closed state of the patient's eye EYE, but not limited to this.

For example, when the patient's eye EYE is in the open state and gazes the projection light source, the regulating unit will regulate the projection light source to emit the multi-band light L for a part or all of the retina of the patient's eye EYE; when the patient's eye EYE in the open state does not gaze the projection light source, the regulating unit will regulate the projection light source to emit the multi-band light L through the sclera of the patient's eye EYE; when the patient's eye EYE is in the closed state, the regulating unit will regulate the projection light source to approach or contact the eyelid of the eye EYE of the patient to project the multi-band light L.

In practical applications, when the optical device 1 is designed as a portable/wearable optical device, the positioning module 10, the tracking and locking module 14 and the monitoring module 16 can be independently disposed, or can be integrated or combined with the control module 18 or other mobile platforms, but not limited to this.

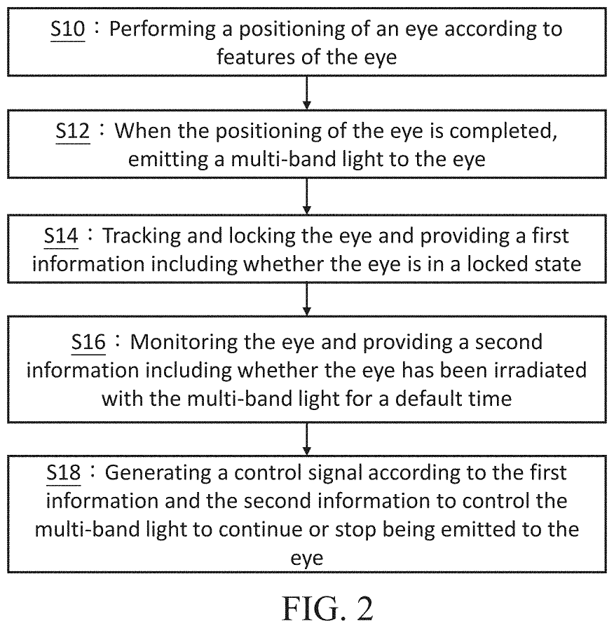

Another embodiment of the invention is an optical device operating method applied to an eye. Please refer to FIG. 2. FIG. 2 illustrates a flowchart of the optical device operating method applied to the eye in this embodiment.

As shown in FIG. 2, the optical device operating method can include the following steps:

Step 10: positioning an eye according to characteristics of the eye;

Step 12: after the eye is positioned, emitting a multi-band light to the eye;

Step 14: tracking and locking the eye and providing a first information including whether the eye is locked;

Step 16: monitoring the eye and providing a second information including whether the eye is irradiated by the multi-band light for a default time; and

Step 18: generating a control signal according to the first information and the second information to continuously emit the multi-band light to the eye or stop emitting the multi-band light to the eye.

In practical applications, when the eyeball of the eye moves slightly and is still in the locked state and the eye has not been irradiated with multi-band light for the default time, the step S18 is to regulate to continue to emit the multi-band light to the eye; when the eyeball of the eye has moved substantially and has been released from the locked state or the eye has been irradiated by the multi-band light for the default time, the step S18 is to regulate to stop emitting the multi-band light to the eye.

In addition, the method can also regulate the multi-band light emitted to the patient's eye in the step S12 according to the open state or the closed state of the patient's eye. For example, when the patient's eye in the open state gazes the projected light source, the method regulates the step S12 to emit the multi-band light to a part or all of the retina of the patient's eye; when the patient's eye in the open state does not gaze the projection light source, the method regulates the step S12 to emit the multi-band light through the sclera of the patient's eye; when the patient's eye is in the closed state, the method regulates the step S12 to emit the multi-band light by approaching or touching the eyelid of the patient's eye.

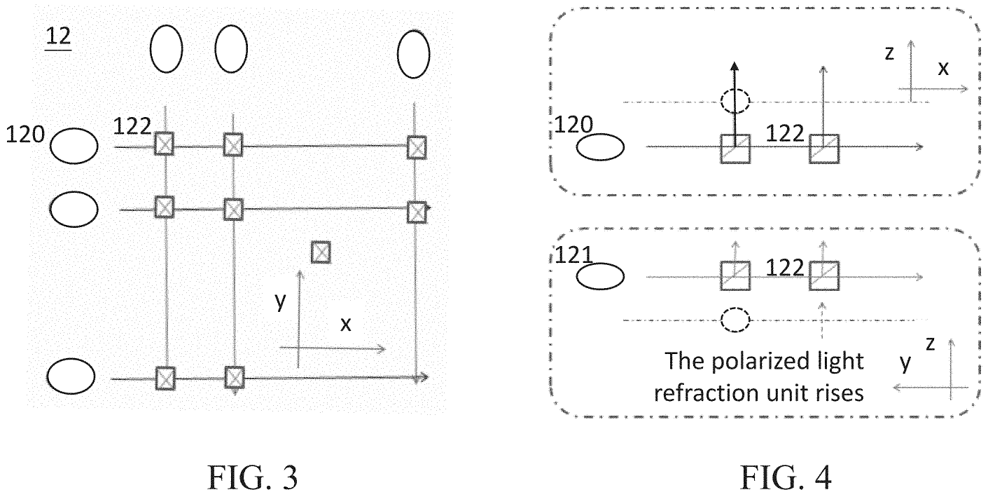

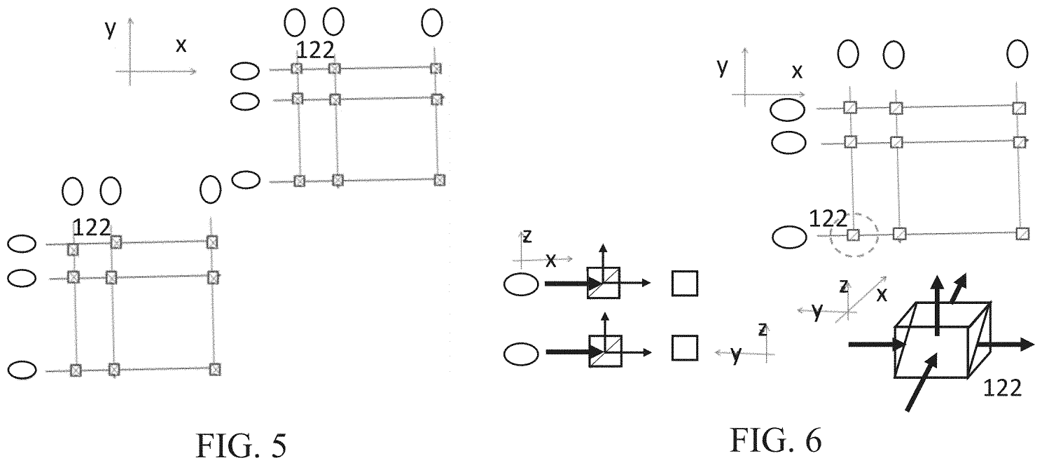

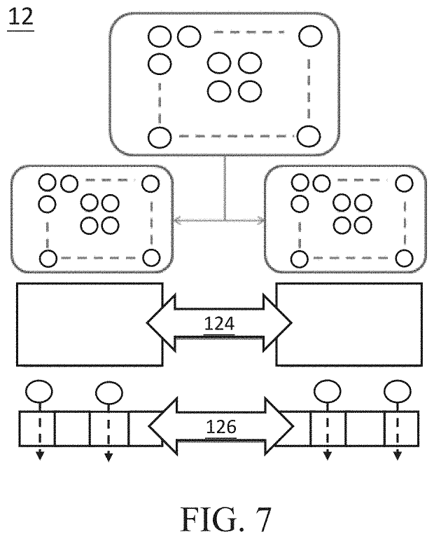

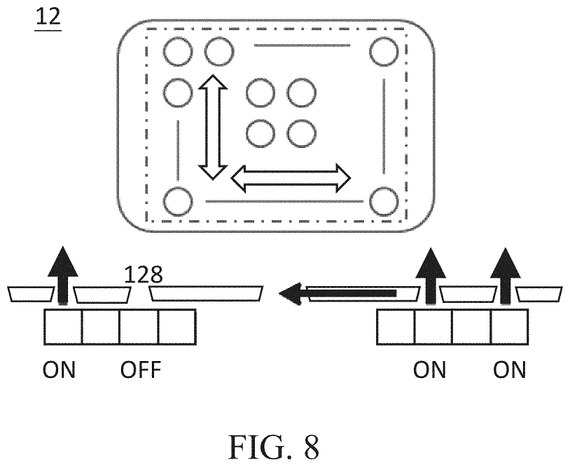

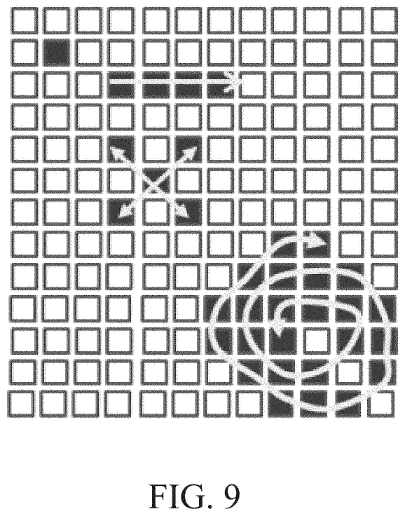

In practical applications, the multi-band light source module 12 can have various design methods of optical paths or optical element structures. For example, as shown in FIG. 3, the multi-band light source module 12 can also include light source units 120 distributed on the edge cooperated with the polarized light refraction unit 122 to provide light projection treatment areas at different positions, but not limited to this; as shown in FIG. 4, when different light source units 120-121 are distributed at different heights, the polarized light refraction unit 122 can provide light projection of different wavelengths through height switching, but not limited to this; as shown in FIG. 5 and FIG. 6, when different light source units are distributed at the same height, the polarized light refraction unit 122 can provide light projection of different wavelengths by shifting or rotating, but not limited to this; as shown in FIG. 7 and FIG. 8, the multi-band light source module 12 can provide light projection of different wavelengths by adding an electrochromic layer 124 and changing the driving voltage, adding a filter 126 and shifting the filter 126, or switching the light source by shifting the aperture layer 128, but not limited to this; the polarized light refraction unit and the light source unit can be independently driven to provide different scanning modes with different shape paths, such as points, lines, radials, spirals, etc. shown in FIG. 9, but not limited to this.

In addition, the type of the light source units 120 used in the multi-band light source module 12 is not particularly limited, and can be laser units, light-emitting diode (LED) units, organic light-emitting diode (OLED) unit, micro light-emitting diode (uLED) unit, etc., and the multi-band light L emitted by the multi-band light source module 12 can have a wavelength of 620-1000 nm, which is equivalent to the wavelength of red light to near-infrared light, but not limited to this.

Compared to the prior art, the optical device and an operating method thereof in the invention can achieve ideal curative effect by projecting lights with different wavelength bands to the fundus of the patient, and can use the retinal blood vessels to locate and track the position change of the patient's eyeball. And, the position of the retina of the irradiation target can still be accurately positioned when moving, and the irradiation can be stopped immediately when the patient's eyeball moves greatly to ensure safety.

With the example and explanations above, the characteristics and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An optical device, comprising:
a positioning module, configured to position an eye according to characteristics of the eye;
a multi-band light-source module, coupled to the positioning module,
configured to emit a multi-band light to the eye after the eye is positioned by the positioning module;
a tracking and locking module, configured to track and lock the eye and provide a first information comprising whether the eye is locked;
a monitoring module, configured to monitor the eye and provide a second information comprising whether the eye is irradiated by the multi-band light for a default time; and
a control module, coupled to the tracking and locking module, the monitoring module and the multi-band light-source module, configured to generate a control signal according to the first information and the second information to control the multi-band light-source module to continuously emit the multi-band light to the eye or stop emitting the multi-band light to the eye.

2. The optical device of claim 1, wherein the optical device is designed as a machine-type optical device.

3. The optical device of claim 1, wherein the optical device is designed as a portable/wearable optical device; the positioning module, the tracking and locking module and the monitoring module are disposed independently, or integrated/combined with the control module and other mobile platforms.

4. The optical device of claim 1, wherein when an eyeball of the eye moves slightly and is still in a locked state and the eye is irradiated by the multi-band light for the default time, the control module controls the multi-band light-source module to continuously emit the multi-band light to the eye.

5. The optical device of claim 1, wherein when an eyeball of the eye moves substantially and is released from a locked state or the eye is irradiated by the multi-band light for the default time, the control module controls the multi-band light-source module to stop emitting the multi-band light to the eye.

6. The optical device of claim 1, wherein the multi-band light-source module comprises a regulating unit and a projection light-source; the regulating unit is configured to regulate the multi-band light emitted by the projection light-source according to an open state or a closed state of the eye.

7. The optical device of claim 6, wherein when the eye is in the open state and the projection light-source is watched by the eye, the regulating unit regulates the projection light-source to emit the multi-band light on a part or all of the retina of the eye; when the eye is in the open state but the projection light-source is not watched by the eye, the regulating unit regulates the projection light-source to project the multi-band light through the sclera of the eye.

8. The optical device of claim 6, wherein when the eye is in the closed state, the regulating unit regulates the projection light-source, close to or contacting the eyelid of the eye, to project the multi-band light.

9. The optical device of claim 1, wherein an electrochromic layer is attached to the multi-band light source module and the wavelength is changed by changing the driving voltage, or a filter is attached and the wavelength is changed by moving the filter, or the light source is switched by moving an aperture layer.

10. The optical device of claim 1, wherein the multi-band light source module comprises a plurality of light source units distributed on the edge and a polarized light refraction unit to provide light projection treatment areas at different positions.

11. The optical device of claim 1, wherein when the plurality of light source units is distributed in different height layers, the polarized light refraction unit provides light projection of different wavelengths by height switching.

12. The optical device of claim 1, wherein when the plurality of light source units is distributed on the same height layer, the polarized light refraction unit provides light projection of different wavelengths by translation or rotation.

13. The optical device of claim 10, wherein when the plurality of light source units is distributed on the same height layer, the polarized light refraction unit provides light projection of different wavelengths by translation or rotation.

14. The optical device of claim 1, wherein the characteristics of the eye are retinal blood vessels.

15. An optical device operating method, comprising following steps of:
(a) positioning an eye according to characteristics of the eye;
(b) after the eye is positioned, emitting a multi-band light to the eye;
(c) tracking and locking the eye and providing a first information comprising whether the eye is locked;
(d) monitoring the eye and providing a second information comprising whether the eye is irradiated by the multi-band light for a default time; and
(e) generating a control signal according to the first information and the second information to continuously emit the multi-band light to the eye or stop emitting the multi-band light to the eye.

16. The optical device operating method of claim 15, wherein when an eyeball of the eye moves slightly and is still in a locked state and the eye is irradiated by the multi-band light for the default time, the step (e) continuously emits the multi-band light to the eye.

17. The optical device operating method of claim 15, wherein when an eyeball of the eye moves substantially and is released from a locked state or the eye is irradiated by the multi-band light for the default time, the step (e) stops emitting the multi-band light to the eye.

18. The optical device operating method of claim 15, further comprising a step of:
regulating the multi-band light emitted by the step (b) according to an open state or a closed state of the eye.

19. The optical device operating method of claim 18, further comprising steps of:
when the eye is in the open state and the projection light-source is watched by the eye, regulating the step (b) to emit the multi-band light on a part or all of the retina of the eye; and
when the eye is in the open state but the projection light-source is not watched by the eye, regulating the step (b) to project the multi-band light through the sclera of the eye.

20. The optical device operating method of claim 18, further comprising a step of:
when the eye is in the closed state, regulating the step (b), close to or contacting the eyelid of the eye, to project the multi-band light.
